(19) United States
(12) Patent Application Publication
(10) Pub. No.: US 2009/0088417 A1
(43) Pub. Date: Apr. 2, 2009

(54) VANILLOID RECEPTOR ANTAGONIST FOR PREVENTION AND TREATMENT OF DRUG DEPENDENCE AND ADDICTION

(76) Inventors: Choon-Gon Jang, Gyeonggi-do (JP); Seok-Yong Lee, Seoul (KR); In-Jee You, Gyeonggi-do (KR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/281,937

(22) PCT Filed: Feb. 15, 2007
(86) PCT No.: PCT/KR07/00825

§ 371 Date: Sep. 5, 2008

(30) Foreign Application Priority Data
Mar. 8, 2006 (KR) 10-2006-0021683

(51) Int. Cl. A61K 31/55; A61P 25/30; A61P 25/36

(52) U.S. Cl. 514/213.01

(57) ABSTRACT

Disclosed is a composition for preventing and treating harmful actions caused by drugs of abuse. More particularly, disclosed is a pharmaceutical composition effective for preventing or treating dependence and spontaneous movements by using a drug acting against the vanilloid receptor Capsazepine, which is used as a drug acting against the vanilloid receptor, shows an excellent effect of alleviating conditions of cocaine dependence and addition during the development and recurrence thereof. Administration of the drug acting against the vanilloid receptor provides important information in developing an agent for preventing and treating drug dependence and addiction.

### TECHNICAL FIELD

The present invention relates to a composition for preventing and treating harmful actions caused by “drugs of abuse”. More particularly, the present invention relates to a pharmaceutical composition effective for preventing or treating dependence and spontaneous movements by using a drug acting against the vanilloid receptor. According to the present invention, capsazepine, which is used as a drug acting against the vanilloid receptor, shows an excellent effect of alleviating conditions of cocaine dependence and addiction during the development and recurrence thereof.

### BACKGROUND ART

In general, activities of the vanilloid receptor relate to diseases such as acute and chronic pain, neuropathic pain, post-surgery pain, migraine, arthralgia, or the like. There have been many studies to treat pain conditions by effectively controlling the vanilloid receptor.

Although capsazepine has been known as a competitive antagonist of the vanilloid receptor, it has been reported that capsazepine does not provide an analgesic or anti-inflammatory effect (see [Perkins and Campbell, 1992, Br. J. Pharmacol. 107, pp 329-333]). However, recent studies have revealed that capsazepine is effective for providing analgesic actions through an animal test (see [Kwak et al., 1998, Neurosci. 86, pp 619-626; Santos and Calixto, 1997, Neurosci. Lett. 235 pp 73-76]). Thus, most previous studies related to the vanilloid receptor deal with treatment of pain conditions.

Meanwhile, as a pharmaceutical composition for treating drug addiction, a composition comprising melatonin for alleviating conditions of drug addiction or benzodiazepine dependence has been developed (Korean Patent Publication No. 10-0425045). Also, novel use of tauroursodeoxycholic acid in treating drug addiction has been disclosed (Korean Patent Publication No. 0129799).

In addition, a method for alleviating drug addiction and abuse tendency by using a vanilloid receptor-1 agonist is disclosed (PCT/US03/12496).

“Drugs of abuse” refers to drugs causing psychological dependence that leads to repeated use of a drug, and particular examples thereof include cocaine, nicotine, morphine, morphine derivatives, methamphetamine, methamphetamine derivatives, heroin, MDMA (3,4-methylenedioxymethamphetamine; ecstasy), dextromethorphan, etc. Such drugs cause psychological excitation, main symptoms of which include hallucination or delusion, when used in a relatively high dose or used repeatedly for a long period of time even in a low dose. Such appearance of psychological intoxication is enhanced by repetitive use. Finally, a very strong physical or psychological dependence of such drugs is developed. In other words, main features of drugs of abuse include excitation of the central nervous system and a reinforcement effect that is a strong liking or need for repeated drug-taking. Therefore, repeated administration of drugs of abuse enhances spontaneous movements and causes drug dependence. Particularly, repeated administration of drugs of abuse exhausts dopamine and reduces activities of dopamine-acting nerves, resulting in a compensative mechanism thereof including activation of the dopamine nervous system at the post-synapse. Therefore, dopamine receptor hypersensitivity and enhancement of spontaneous movements are developed, resulting in a strong need to use such drugs repeatedly. Due to the effect of psychological excitation caused by such drugs, persons depending on and addicted to such drugs are increasing in number from day to day. This leads to serious social problems, and thus there is an imminent need for an agent for preventing and treating such drug addicts.

Under these circumstances, the inventors of the present invention have found that capsazapine, which is a vanilloid receptor antagonist, has an effect of alleviating conditions of drug dependence and addiction. The present invention is based on this finding.

### DISCLOSURE OF INVENTION

### Technical Problem

It is an object of the present invention to provide a pharmaceutical composition having an excellent effect of alleviating conditions of drug dependence and addiction.

It is another object of the present invention to provide a method for alleviating conditions in a patient suffering from drug dependence and addiction.

### Technical Solution

In order to achieve the above-mentioned objects, a vanilloid receptor antagonist is used to prevent and treat drug dependence and addiction according to the present invention.

In the present invention, a conditioned place preference (CPP) test is used to monitor the degree of alleviation in drug dependence and addiction. The CPP system used in the present invention includes a white box, a black box and a pathway connecting the two boxes with each other. Mice are used as test animals and effects on preference/aversion are analyzed by measuring residence times of the mice in the two boxes.

To perform the test, test animals are divided into a physiological saline administration group, a cocaine administration group and a capsazepine+cocaine administration group.

According to an aspect of the present invention, there is provided a composition for preventing and treating development and recurrence of drug dependence and addiction, the composition comprising a vanilloid receptor antagonist as an active ingredient. Preferably, the composition according to the present invention comprises capsazepine, which is a vanilloid receptor antagonist, as an active ingredient.

The pharmaceutical composition according to the present invention may be administered in various parenteral forms. When converting the pharmaceutical composition into administration forms, conventional diluents or excipients such as fillers, extenders, binders, wetting agents, disintegrating agents or surfactants are used. Such parenteral formulations include sterilized aqueous solution, non-aqueous solution, suspension, emulsion, freeze-dried formulations, and suppositories. As a solvent for non-aqueous solution and suspension, propylene glycol, polyethylene glycol, vegetable oil such as olive oil, or injectable ester such as ethyolate may be used. As a base for suppositories, Witepsol, Macrogol, Tween 61, cacao fat, laurin fat, glycerogelatin, etc. may be used.

Additionally, the composition for preventing and treating development and recurrence of drug dependence and addiction may further comprise various pharmaceutically acceptable carriers such as physiological saline or organic solvents. Also, in order to increase the stability or absorptivity, carbohydrates such as glucose, sucrose or dextran, antioxidants such as ascorbic acid or glutathione, chelating agents, low-molecular weight proteins or other stabilizers may be used.

As used herein, the term “pharmaceutically acceptable carriers” means at least one compatible solid or liquid filler, diluent or encapsulation material suitable to be administered to a subject. Herein, “compatible” is a term that refers to the pharmaceutically acceptable carriers harmonized with the vanilloid receptor antagonist or with each other so that no interaction may occur to substantially reduce the pharmaceutical effects according to the pharmaceutical composition of the present invention under the conditions of conventional applications. It is a matter of course that the pharmaceutically acceptable carriers have a sufficiently high purity and a sufficiently low toxicity suitable to be administered to a subject in need of treatment.

The pharmaceutical composition according to the present invention may be administered to a subject in an effective dose of 0.1˜100 mg/kg, preferably 1˜10 mg/kg, once to three times per day.

The total effective dose of capsazepine in the pharmaceutical composition according to the present invention may be administered to a patient via bolus injection or via infusion for a relatively short period of time in a single dose, or may be administered for a long period of time in multiple doses according to a fractionated treatment protocol. The effective dose is determined considering various factors including mode of administration, treatment frequency, age and physical conditions of the patient, etc. In view of this, those skilled in the art may easily determine an effective dose suitable for the particular use of the pharmaceutical composition according to the present invention.

### ADVANTAGEOUS EFFECTS

The pharmaceutical composition according to the present invention, which comprises capsazapine as a vanilloid receptor antagonist, has an effect of alleviating conditions of drug dependence and addiction.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a graph showing the results of a test for determining the effect of capsazepine upon the inhibition of development of psychological dependence on cocaine by measuring residence times in CPP (conditioned place preference) boxes for four test groups (it can be seen that administration of capsazepine as a vanilloid receptor antagonist significantly inhibits development of dependence on cocaine);

FIG. 2 is a graph showing the results of a test for determining the effect of capsazepine upon inhibition of recurrence of psychological dependence on cocaine by measuring residence times in CPP boxes for four test groups (it can be seen that single-dose pre-treatment administration of capsazepine as a vanilloid receptor antagonist significantly inhibits recurrence of dependence on cocaine after a withdrawal of cocaine);

FIG. 3 is a graph showing the results of a test for determining the effect of capsaicin upon augmentation in development of psychological dependence on cocaine by measuring residence times in CPP boxes for four test groups (it can be seen that administration of capsaicin as a capsaicin receptor agonist increases dependence on cocaine); and

FIG. 4 is a series of graphs showing the results of the effect of capsazepine upon inhibition of behavioral sensitization in recurrence of psychological dependence on cocaine by measuring displacement from spontaneous movements (it can be seen that administration of capsazepine as a capsaicin receptor antagonist significantly inhibits behavioral sensitization caused by cocaine).

### MODE FOR THE INVENTION

Reference will now be made in detail to the preferred embodiments of the present invention. However, the following examples are illustrative only, and the scope of the present invention is not limited thereto. All documents cited herein are incorporated by reference.

### EXAMPLES

### Example 1

### Effect of Capsazepine Upon Inhibition of Development of Psychological Dependence on Cocaine

Development of psychological dependence on cocaine was determined according to the CPP (conditioned place preference) test. See [Calcagnetti and Schechter 1993, Physiol Behav 51:667-672; Carr et al. 1989, Clarendon Press, Oxford, pp 264-319; M. T. Bardo, R, A. Bevins (2000) Conditioned place preference: what does it add to our preclinical understanding of drug reward? Psychopharmacology 153:31-431].

1. Test System

The following CPP system was used to perform the test. The CPP system included two boxes, each having a dimension of 15 cm×15 cm×15 cm (width×depth×height). One of the boxes was made of white acryl sheets on three sides thereof, while the other was made of black acryl sheets on three sides thereof. Each box had a front surface made of a transparent acryl sheet. Between the two boxes, a gray-colored pathway having a dimension of 3×3×7.5 cm was provided. Also, a Guillotine door capable of interrupting the pathway was provided. Further, the bottom surface of the white box was rough and that of the black box was smooth, so that mice had a different contact feel between the two boxes. The test was performed under an illuminance maintained at 20 Lux.

2. Test Procedure

Step 1 (Basic Test): On the first day of the test, the Guillotine door between the two boxes was opened, and a male ICR mouse having a body weight of 20˜25 g made available by MJ Ltd. (Seoul) was accommodated in the CPP system so that it could move freely therein for 5 minutes. On the second day of the test, the mouse was introduced into the CPP system in the same manner as the first day. Then, residence times of the mouse in the two boxes were measured for 15 minutes to obtain reference values.

Step 2 (Conditioning Step): On the third, the fifth and the seventh days of the test, the Guillotine door between the two boxes was closed, and cocaine made available by Macfarlan Smith Ltd. (England) was administered to the mouse in a dose of 15 mg/kg via intraperitoneal injection. Next, the mouse was allowed to remain in the white box, for which it showed an aversion, for 40 minutes. Meanwhile, on the fourth, the sixth and the eighth days of the test, physiological saline was administered to the same mouse, and the mouse was allowed to remain in the black box, for which it showed a preference, for 1 hour. Further, capsazepine (made available by Tocris) as a competitive inhibitor to the vanilloid receptor was administered to the mouse in a dose of 5 mg/kg and 2.5 mg/kg via an intraperitoneal administration, 30 minutes before the administration of cocaine (15 mg/kg).

Step 3 (Test Step): On the ninth day of the test, the Guillotine door between the two boxes was opened, a pathway was provided in the middle of the boxes, and then a mouse, to which no drug was administered, was allowed to remain in the two boxes for 15 minutes. Then, residence times in the white box and in the black box were measured and compared to the values measured on the second day of the test. The degree of development of psychological dependence was calculated by subtracting the reference values from the measured values.

Therefore, test animals were divided into four groups (each group having ten mice), wherein physiological saline was administered to the first test group, cocaine was administered to the second test group, cocaine +2.5 mg/kg of capsazepine (pretreatment) was administered to the third test group, and cocaine +5.0 mg/kg of capsazepine (pretreatment) was administered to the fourth test group. The test animals were accommodated in the CPP system, and residence times in the CPP boxes were measured on the ninth day of the test.

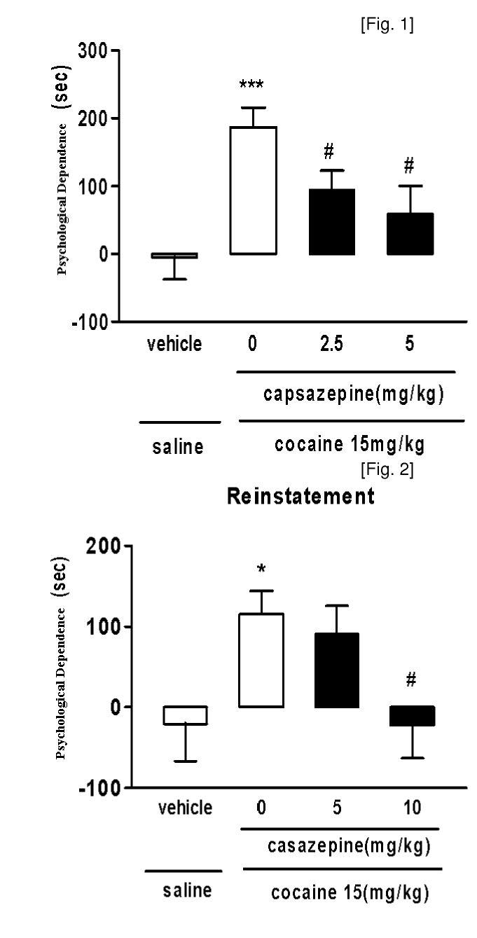

As a result, the control group, to which cocaine was administered, showed a significant psychological dependence (p<0.001). During the accommodation period, the test groups treated with cocaine+capsazepine displayed inhibited development of psychological dependence to substantially the same degree obtained from the test group treated with saline. This indicates that capsazepine significantly inhibits development of psychological dependence on cocaine (p<0.05) (FIG. 1). In FIG. 1,*** represents p<0.001, a significant result being obtained as compared to the control group treated with saline (vehicle), while # represents p<0.05, a significant result being obtained as compared to the test group treated with cocaine.

### Example 2

### Effect of Capsazepine Upon Inhibition of Recurrence of Psychological Dependence on Cocaine

Test animals were divided into four groups. Psychological saline was administered to the first test group (10 mice), and cocaine was administered to the remaining second test group, third test group and fourth test group (30 mice in total). On the ninth day of the test, development of a dependence on cocaine was checked, and then saline was administered alone for the subsequent 5 days to allow the mice to lose the cocaine dependence. On the fifteenth day of the test, saline was administered to the first test group, cocaine was administered to the second test group, 5 mg/kg of capsazepine (pretreatment)+cocaine was administered to the third test group, and 10 mg/kg of capsazepine (pretreatment)+cocaine was administered to the fourth test group, so as to allow recurrence of psychological dependence on cocaine. Residence times in the CPP boxes were measured as described above.

As a result, the control group treated with cocaine showed recurrence of a strong dependence on the drug (p<0.05), while the test group pre-treated with 10 mg/kg of capsazepine showed inhibition of such recurrence (p<0.05) (FIG. 2). In FIG. 2, *** represents p<0.05, a significant result being obtained as compared to the control group treated with saline, while # represents p<0.05, a significant result being obtained as compared to the test group treated with cocaine.

### Example 3

### Effect of Capsaicin Upon Increase of Development of Psychological Dependence on Cocaine

As described in Example 1, test animals were divided into four groups (each group having ten mice). Physiological saline was administered to the first test group, saline +0.3 mg/kg of capsaicin (pretreatment) was administered to the second test group, and cocaine was administered to the third test group, and cocaine +0.3 mg/kg of capsaicin (pretreatment) was administered to the fourth test group. The test animals were accommodated in the CPP system, and residence times in the CPP boxes were measured on the ninth day of the test.

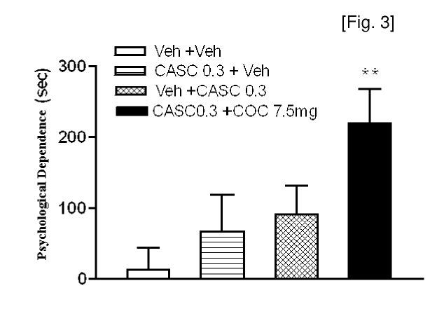

As a result, the test group, to which cocaine +0.3 mg/kg of capsaicin (pretreatment) was administered, showed a significant increase in psychological dependence on cocaine (p<0.01). This indicates that capsaicin increases psychological dependence on cocaine. In the test, the displacement from the movements of the animals between the two boxes was measured for 15 minutes (FIG. 3). In FIG. 3, *** represents p<0.01, a significant result being obtained as compared to the control group treated with saline.

### Example 4

### Effect of Capsazepine Upon Behavioral Sensitization in Recurrence of Psychological Dependence on Cocaine

Behavioral sensitization in recurrence of psychological dependence on cocaine was determined according to the locomoter activity. See [Yoo et al. 2003, Neurosci Lett. 433, pp 37-40].

Test animals were divided into four groups as follows: physiological saline was administered to the first test group, cocaine was administered to the second test group, cocaine +2.5 mg/kg of capsazepine (pretreatment) was administered to the third test group, and cocaine +5.0 mg/kg of capsazepine (pretreatment) was administered to the fourth test group.

(1) Test System

A locomotor box was used for the test. The locomotor box had a dimension of 30 cm×30 cm×30 cm (width×depth×height) and included wall surfaces formed of dark colored acryl sheets. The bottom of the box was covered with a black sheet having substantially the same size as the former.

(2) Test Procedure

Step (1)—Basic Test: On the first day, mice were allowed to move freely in the locomotor box for 30 minutes so that they were accommodated.

Step (2)—Development of Behavioral Sensitization:

After the accommodation period, 15 mg/kg of cocaine was administered to the mice via intraperitoneal injection, and then the mice were allowed to remain in the locomotor box. The displacement from spontaneous movements of the mice was measured. Then, the same dose of drug was repeatedly administered to the mice till the fifth day of the test, and the mice were introduced into a home cage. No measurement was performed at this time. On the sixth day of the test, the drug was administered again to the mice and the displacement from spontaneous movements was measured. For the subsequent five days, no drug was administered. On the twelfth day of the test, the same dose of cocaine was administered to the test animals of the whole test groups. Then, displacement from spontaneous movements was measured. Measurement of displacement from spontaneous movements of the mice was carried out for 60 minutes in the locomotor box according to a video-tracking method using the NeuroVision program (Pusan National University, Pusan, Korea). See [Yoo et al. 2004, Neurosci Lett. 433, pp 37-40].

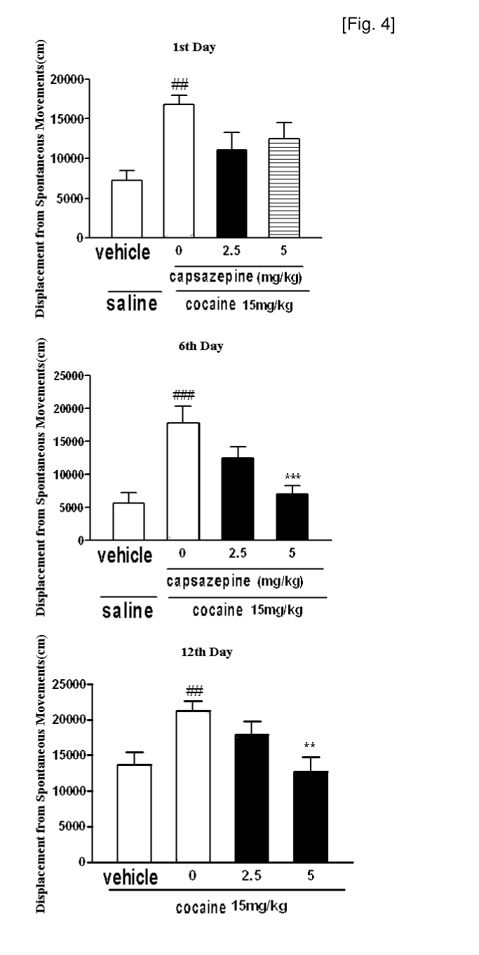

As a result, the control group treated with cocaine showed a significant increase in spontaneous movements and development of a strong behavioral sensitization phenomenon on the sixth day of the test due to the repeated administration of cocaine (p<0.01). On the contrary, such behavioral sensitization was inhibited in the test group pre-treated with 5 mg/kg of capsazepine (p<0.001). Repeated administration of the same dose of cocaine carried out after a drug withdrawal period of five days caused conditions of cocaine addiction in the control group treated with cocaine (p<0.01). As compared to the control group, behavioral sensitization caused by cocaine was completely inhibited in the test group pre-treated with 5 mg/kg of capsazepine to the same degree obtained from the control group treated with saline (p<0.01) (FIG. 4). In FIG. 4, ## represents p<0.01 and ### represents p<0.001, significant results being obtained as compared to the control group treated with saline, while ** represents p<0.01 and *** represents p<0.001, significant results being obtained as compared to the test group treated with cocaine.

The pharmaceutical composition according to the present invention may be provided in the following forms. However, the scope of the present invention is not limited to the following formulation examples.

### Formulation Example 1

### Powder

Powder of capsazepine or a compound thereof 20 mg

Lactose 100 mg

Talc 10 mg

The above ingredients were mixed and packed into a sealed bag to provide powder formulations.

### Formulation Example 2

### Tablets

Powder of capsazepine or a compound thereof 10 mg

Corn starch 100 mg

Lactose 100 mg

Magnesium stearate 2 mg

The above ingredients were mixed and formed into tablets according to a conventional method for forming tablets to provide tablet formulations.

### Formulation Example 3

### Capsules

Powder of capsazepine or a compound thereof 10 mg

Crystalline cellulose 3 mg

Lactose 14.8 mg

Magnesium stearate 0.2 mg

The above ingredients were mixed and filled into gelatin capsules according to a conventional method for forming capsules to obtain capsule formulations.

### Formulation Example 4

### Injection Formulations

Powder of capsazepine or a compound thereof 10 mg

Mannitol 180 mg

Sterilized distilled water for injection 2,974 mg

Na₂HPO₄.12H₂O 26 mg

According to a conventional method for forming injection formulations, the above ingredients were used per ampoule (2 ml).

### Formulation Example 5

### Liquid Formulations

Powder of capsazepine or a compound thereof 20 mg

Fructose 10 g

Mannitol 5 g

Purified water q.s.

Each of the above ingredients was dissolved into purified water according to a conventional method for forming liquid formulations. Next, an adequate amount of lemon flavor was added thereto, the ingredients were thoroughly mixed, and purified water was added q.s. to 100 ml. Then, the mixed ingredients were filled into brown bottles and sterilized to provide liquid formulations.

### INDUSTRIAL APPLICABILITY

As can be seen from the foregoing, according to the present invention, capsazepine as a vanilloid receptor antagonist can be used to prevent and treat development and recurrence of drug dependence and addiction.

Although several preferred embodiments of the present invention have been described for illustrative purposes, those skilled in the art will appreciate that various modifications, additions and substitutions are possible, without departing from the scope and spirit of the invention as disclosed in the accompanying claims.

1. A composition for preventing and treating drug dependence and addiction, which comprises a vanilloid receptor antagonist as an active ingredient.

2. The composition for preventing and treating drug dependence and addiction as claimed in claim 1, wherein the vanilloid receptor antagonist is capsazepine.

3. The composition for preventing and treating drug dependence and addiction as claimed in claim 1 or claim 2, wherein the drug causing the drug dependence and addiction is at least one drug selected from the group consisting of cocaine, methamphetamine, nicotine, morphine, heroin, MDMA (3,4-methylenedioxymethamphetamine; ecstasy) and dextromethorphan.

4. The composition for preventing and treating drug dependence and addiction as claimed in claim 1 or 2, which further comprises pharmaceutically acceptable carriers.

5. The composition for preventing and treating drug dependence and addiction as claimed in claim 3, which further comprises pharmaceutically acceptable carriers.

6. A composition for preventing and treating recurrence of drug dependence and addiction, which comprises a vanilloid receptor antagonist as an active ingredient.

7. The composition for preventing and treating recurrence of drug dependence and addiction as claimed in claim 6, wherein the vanilloid receptor antagonist is capsazepine.

8. The composition for preventing and treating recurrence of drug dependence and addiction as claimed in claim 7 or 8, wherein the drug causing the drug dependence and addiction is at least one drug selected from the group consisting of cocaine, methamphetamine, nicotine, morphine, heroin, MDMA (3,4-methylenedioxymethamphetamine; ecstasy) and dextromethorphan.

9. The composition for preventing and treating recurrence of drug dependence and addiction as claimed in claim 6 or 7, which further comprises pharmaceutically acceptable carriers.

10. The composition for preventing and treating recurrence of drug dependence and addiction as claimed in claim 8, which further comprises pharmaceutically acceptable carriers.
